(19) United States
(12) Patent Application Publication
(10) Pub. No.: US 2011/0086376 A1
(43) Pub. Date: Apr. 14, 2011

(54) In-Vivo Platelet Function Test By Online Bleeding Volume Measurement

(76) Inventor: Jeffrey A. Klein, San Juan Capistrano, CA (US)

(21) Appl. No.: 12/579,105

(22) Filed: Oct. 14, 2009

(51) Int. Cl. C12Q 1/02

(52) U.S. Cl. 435/29

(57) ABSTRACT

A method for remotely determining a patient's excessive bleeding tendency and a patient's resistance to blood thinning medication is disclosed. An incision is made in the patient's forearm. Blood oozing out of the incision is absorbed into a blotter paper until the bleeding stops. Blotches of blood formed on the blotter paper are captured as an image and sent to a service provider who calculates a value associated with the bleeding volume of the patient. The service provider retransmits a value associated with the bleeding volume back to the medical professional. To determine the resistance to blood thinning medication, one incision is made in the patient prior to administration of blood thinning medication. Blood oozing out of the incision is collected on blotter paper until the patient stops bleeding. A second incision is made in the patient. A second set of blotter paper is used to collect the blood oozing out of the incision until the bleeding stops. Both sets of blotter paper are sent to a service provider to calculate a value associated with the difference in bleeding volume. The service provider then retransmits the value associated with the difference in bleeding volume to the medical professional.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND

The present invention relates to a method and apparatus of remotely determining bleeding volume and determining resistance of a patient to blood thinning medication.

There is a need for a general in-vivo screening test for the initial evaluation of patient's with respect to possible bleeding disorders. There are several commercially available in-vitro tests which can detect specific single factor aspects of bleeding disorder. However, bleeding disorders are typically multi-factorial and therefore the in-vitro test may not take into account all of the factors involved in evaluating a patient's possible bleeding disorders.

Bleeding disorders may include excessive bleeding tendency. As such, the patient may be susceptible to bleeding during an operation. Preoperative detection of excessive bleeding tendencies would be helpful in determining whether a potential operation would be more beneficial to a patient in light of its potential risks.

Bleeding disorders are often associated with platelet dysfunction or imbalance. Platelets, also known as thrombocytes, are the cell fragments circulating in the blood and are involved in the formation of blood clots. Either platelet dysfunction (malfunction) or low level of platelets (low concentration of platelets in the blood) may increase the risk of bleeding, while high levels of platelets may increase the risk of blood clot formation. Platelets are tiny sized fragments derived from megakaryocyte cells in the bone marrow. Platelets contain granules known as dense granules. Dense granules contain adenine diphosphate (ADP), ADP ionized calcium, histamine, serotonin, and epinephrine. When platelet is activated by contact with injured blood vessels, it degranulates, releasing the contents of the dense granules which cause other platelets to clump together and start the clotting process. As such, it would be beneficial to know whether the patient has a low number of platelets or platelet dysfunction.

Another blood related disorder is related to a patient's resistance to blood thinning (anti-thrombotic or anti-clotting) medication. Resistance to blood thinning medication or, in other words, aspirin resistance or aspirin non-responsiveness may be defined as the failure of blood thinning medication (e.g., aspirin) to inhibit platelet function as determined by an in-vitro test (“in a test tube” test). Estimates of the prevalence of aspirin resistance (the estimated percentage of people with aspirin resistance) vary widely among studies and may depend upon the type of platelet function test being used. Estimates of the prevalence of aspirin resistance among healthy patients vary from five (5) percent to fifty (50) percent. Patients who demonstrate aspirin resistance with one test often do not exhibit aspirin resistance with another test. Thus, it appears that aspirin resistance does not appear to be well defined and well characterized. Hence, a need exists for an in-vivo means of assessing aspirin resistance.

The bleeding time of a patient, which is the length of time blood continues to flow after the skin of a patient has been punctured or cut, has been used to identify patients having abnormal bleeding tendencies or platelet dysfunction or low number of platelets. The bleeding time test is an in-vivo test which involves making a standardized incision on the skin then applying a round disc of standardized blotter paper to the enlarging blood droplet adjacent to the incision for precisely thirty (30) seconds, then rotating the blotter paper and applying a fresh edge of the disc to the bleeding incision, and repeating this process until bleeding has stopped. The sequential blotches of blood on the blotter paper should not intersect. The total number of dried blotches of blood on the blotter paper divided by the number two (2) gives the duration of bleeding in minutes.

For many years the bleeding time test was widely used as a pre-surgical test to predict the risk of excessive surgical bleeding. However, based on several comprehensive reviews in the 1990's, the bleeding time test is now regarded as insensitive and has been largely abandoned as a routine pre-operative clinical test. Nevertheless, the bleeding time test remains the only readily available in-vivo test for platelet function or inadequate platelet numbers. One criticism of the bleeding time test is that it has insufficient sensitivity and specificity. For example, it is often desirable to avoid surgery on a patient who is taking aspirin because of the risk of excessive surgical bleeding. The mean bleeding time of persons who take aspirin and those that take no medication do not differ by more than two (2) standard deviations. Thus, the bleeding time test cannot reliably distinguish between persons who take aspirin and those who do not.

Moreover, there are several factors which can affect the results and consistency of a bleeding time test including Von Willebrand Disease, alcohol, temperature, cirrhosis, and renal insufficiency. Mild systemic hypothermia also influences platelet adhesion and aggregation and coagulation reaction. Bleeding time is also sensitive to alterations of skin temperature. By way of example and not limitation, lowering the skin temperature from thirty-two (32) degrees Celsius to between thirty (30) degrees Celsius and twenty-eight (28) degrees Celsius with a preserved core temperature can more than double the bleeding time. Hence, the bleeding time test does not appear to be a reliable test in determining a patient's potential bleeding disorder or platelet dysfunction or inadequate platelet numbers.

Aspirin is believed to be very effective in preventing acute myocardial infarction and major stroke. As a result of multiple clinical trials demonstrating the anti-platelet and antithrombotic effect of aspirin, it is now the primary treatment for prevention of heart attack and stroke. However, there is recent evidence that some subset of the population is resistant to the beneficial effects of aspirin. Patients with aspirin resistance who take aspirin prophylactically may not receive a significant benefit yet are exposed to the risk of aspirin side effects such as gastric irritation or erosion.

Current diagnostic clinical laboratory devices for detecting the conditions discussed above may cost more than $9,000 plus disposable supplies. The costs to operate these devices may include waste due to expired perishable reagents, expensive cartridges and/or other expensive disposable supplies. As such, barriers to entry exist for these current devices. Moreover, in remote and/or impoverished areas of the world, purchasing, installing and operating such an expensive device may be cost inefficient. As such, medical professionals in these areas may treat patients without the benefits and additional information which may be necessary to properly treat the patient.

For the foregoing reasons, there is a need in the art for an improved in-vivo test to determine both excessive bleeding tendencies of a patient and resistance to blood thinning medication.

### BRIEF SUMMARY

A medical professional in a remote location can now determine a patient's bleeding tendency as long as the medical professional has the means of capturing an image and transmitting the captured image to a service provider who calculates a value associated with the bleeding volume then retransmits (i.e., returns) such value back to the medical professional. The medical professional may use the value associated with the bleeding volume to make an educated treatment decision for that particular patient. In particular, the medical professional performs a traditional bleeding time test by making an incision on the forearm of the patient while a blood pressure cuff is mounted to an upper arm portion of the patient and set to a predetermined pressure (e.g., 40 mm Hg). Once the incision is made, blood will ooze out of the incision. Blotter paper is used to absorb accumulating blood adjacent the incision. Every thirty (30) seconds or at every predetermined time interval, the blotter paper is rotated and a clean fresh unused edge of the blotter paper absorbs the accumulating blood such that the absorbed blotches of blood do not intersect. This process is continued until blood stops oozing out of the incision. The surface area of the blotches of blood formed on the blotter paper has a linear relationship with the volume of blood. The medical professional may now capture (e.g., scan, photograph, etc.) the blotter paper with blotches formed thereon and transmit the captured image to the service provider (e.g., internet based server, database server, medical service provider, etc.) who will then calculate a value associated with the bleeding volume of the patient. The service provider will then transmit the value associated with the bleeding volume back to the medical professional such that the medical professional may now make an educated treatment decision for that particular patient.

A method of determining a resistance score of a patient's resistance to blood thinning medication is also disclosed. In particular, a medical professional performs one or more bleeding volume tests on the same patient on two separate occasions as discussed herein, once before the patient receives a blood thinning medication and again after the patient has been given the blood thinning medication. Two sets of blotter papers are collected. The first set represents blood gathered from the patient while the patient does not have blood thinning medication within his/her system. The second set of blotter paper represents blood gathered from the patient after administration of a blood thinning medication. The two sets of blotter papers are transmitted to a service provider who calculates a value associated with a difference in or ratio of some mathematical function of the bleeding volumes of the patient before and after administration of blood thinning medication. The value is then transmitted back to the medical professional such that the medical professional may now make an educated treatment decision for that particular patient.

More particularly, in an embodiment, a method for determining bleeding volume of a patient at a remote location is disclosed. The method may comprise the steps of breaking the skin of the patient to induce bleeding, absorbing blood of the patient on a flat absorbent material until the patient stops bleeding, providing a reference surface area on the flat absorbent material, scanning an image of the flat absorbent material including blotches of the absorbed blood and the reference surface area, transmitting the scanned image to a service provider, and receiving a value associated with the bleeding volume of the patient from the service provider.

The breaking the skin of the patient may include the step of making an incision on the skin of the patient. The flat absorbent material may be a standard blotter paper for conducting a bleeding time test. The scanning step may include the step of creating a portable document file or picture file of the flat absorbent material. The transmitting step may include the step of emailing the scanned image to the service provider. The scanning step and the transmitting step may include the step of faxing an image of the flat absorbent material to the service provider.

In another embodiment, a method for determining bleeding volume of a patient at a remote location is disclosed. The method may comprise the steps of receiving a scanned image of a flat absorbent material including absorbed blood of the patient and a reference surface area, determining the area of absorbed blood, determining a value associated with the bleeding volume of the patient as a function of the area of absorbed blood and the reference surface area, and transmitting the value to the remote location.

The determining the area of the absorbed blood step may include the steps of determining a number of pixels of the absorbed blood on the flat absorbent material, determining a number of pixels of the reference surface area, and dividing the number of pixels of the absorbed blood by the number of pixels of the reference surface area.

The transmitting step may include the step of faxing, emailing, transmitting over the internet, refreshing a website accessed from the remote location, or calling by telephone.

In another embodiment, a method of determining resistance of a patient to blood thinning medication is disclosed. The method may comprise the steps of breaking a skin of the patient to induce bleeding prior to administering blood thinning medication, absorbing the blood of the patient on a first flat absorbent material until the patient stops bleeding, administering the blood thinning medication to the patient, breaking the skin of the patient to induce bleeding after administering the blood thinning medication to the patient for a period of time so that the blood thinning medication achieves maximum effect, absorbing the blood of the patient on a second flat absorbent material until the patient stops bleeding, transmitting one or more images of the first and second flat absorbent material to a service provider, and receiving a value associated with a difference or ratio in bleeding volume prior to and after administration of the blood thinning medication from the service provider based on the one or more images of the first and second flat absorbent material.

The administering step may include any means of drug delivery such as the step of injecting the patient with the blood thinning medication or directing the patient to orally ingest the blood thinning medication.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 illustrates a blotter paper absorbing blood oozing out of an incision on a forearm of a patient;

FIG. 2 illustrates one or more blotter papers with blotches of blood on the blotter paper shown in FIG. 1;

FIG. 2A illustrates a blotter paper with a known quantity of blood absorbed into the blotter paper forming a blotch of blood;

FIG. 3 illustrates a process of remotely obtaining a value associated with a patient's bleeding volume;

FIG. 4 illustrates a process of determining a patient's resistance to blood thinning medication; and

FIG. 5 illustrates a process of remotely obtaining a resistance score associated with a patient's resistance to blood thinning medication.

### DETAILED DESCRIPTION

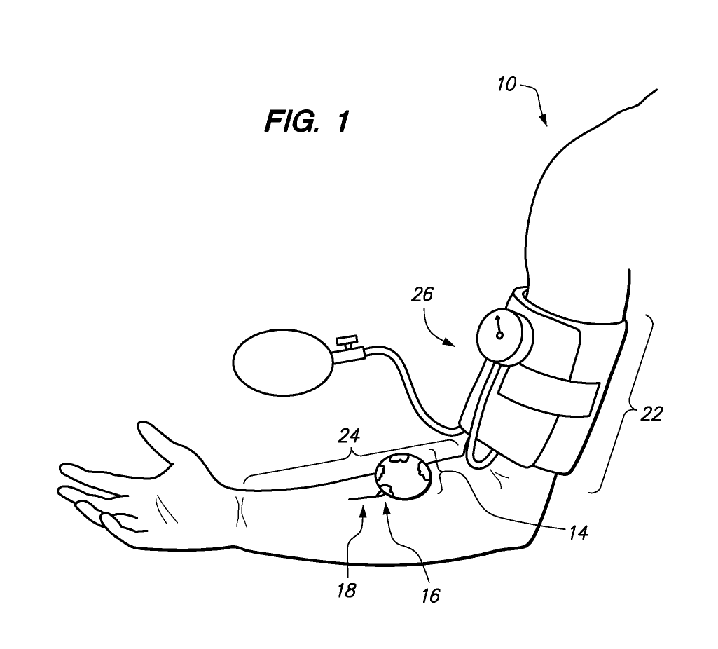

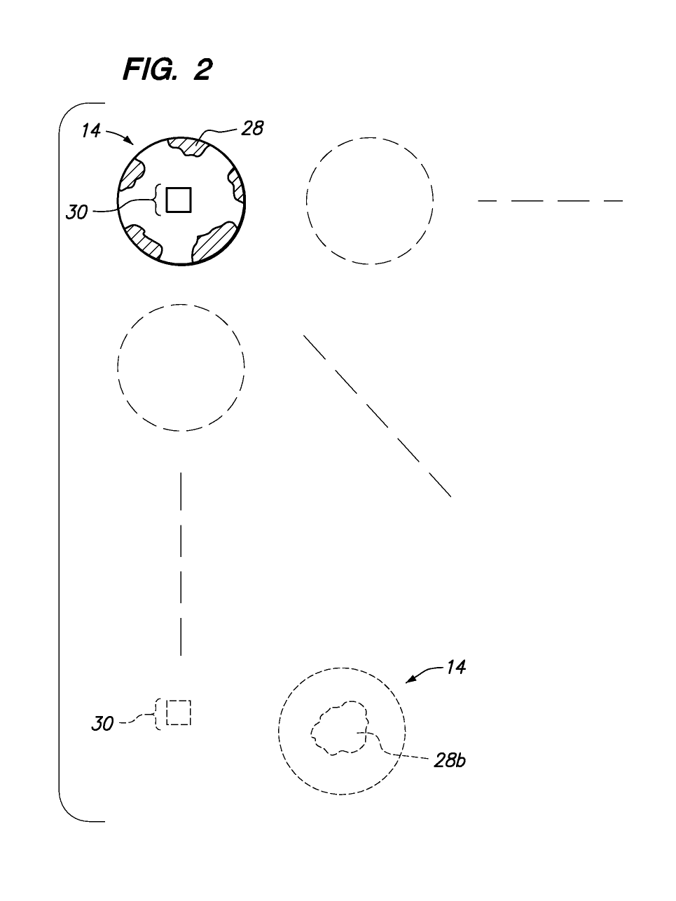

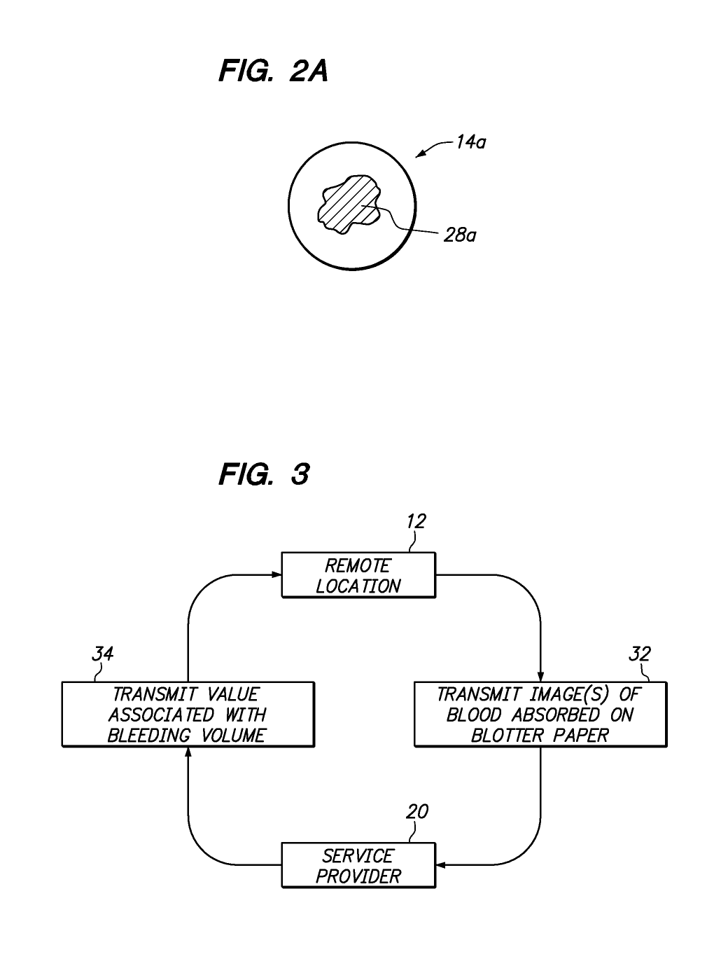

Referring now to the drawings, a method for determining bleeding volume of a patient 10 at a remote location 12 is disclosed. As shown in FIG. 1, a blotter paper 14 is used to absorb discrete blotches of blood 16 oozing out of an incision 18. The blotter paper 14 absorbs the blood 16 oozing out of the incision 18 until the patient 10 stops bleeding. One or more blotter papers 14 may be necessary to absorb all of the blood 16. The blotter paper(s) 14 (see FIG. 2) may be scanned as an image and transmitted 32 to a service provider 20, as shown in FIG. 3. The service provider 20 may be an internet server, database server, a group of medical professionals or a business that provides the calculations, transmissions, information reception and other services discussed herein in relation to the role of the service provider. By way of example and not limitation, the service provider 20 may refer to a website accessible over the internet. The website may provide a means to upload scanned image and submit the scanned image thereby transmitting the scanned image to the service provider 20. The back end of the website may automatically calculate information based on the scanned image and re-transmit information back to the medical professional. Alternatively, the service provider may be a group of technicians or the like that receive the uploaded scanned images, performs calculations and retransmits information back to the medical professional. As such, the service provider may be anyone, device or combination that performs at least one of the functions (e.g., calculates, receives or retransmits information) regarding the service provider discussed herein.

The service provider 20 receives the scanned image and calculates a total blood volume or other bleeding volume based value (e.g., bleeding rate) based on a surface area of the discrete blotches of absorbed blood 16 on the blotter paper 14. The surface area of the discrete blotches 28 of absorbed blood is shown hatched in FIG. 2. The in-vivo bleeding volume test results of the patient 10 is indicative of a patient's excessive bleeding tendencies, platelet function and platelet number. The service provider 20 transmits 34 (see FIG. 3) the bleeding volume based value back to the medical professional at the remote location 12. A medical professional treating the patient 10 at the remote location 12 is now capable of determining the patient's excessive bleeding tendencies as long as the medical professional has access to a scanner and a means for transmitting the scanned image to the service provider 20 such as through the internet.

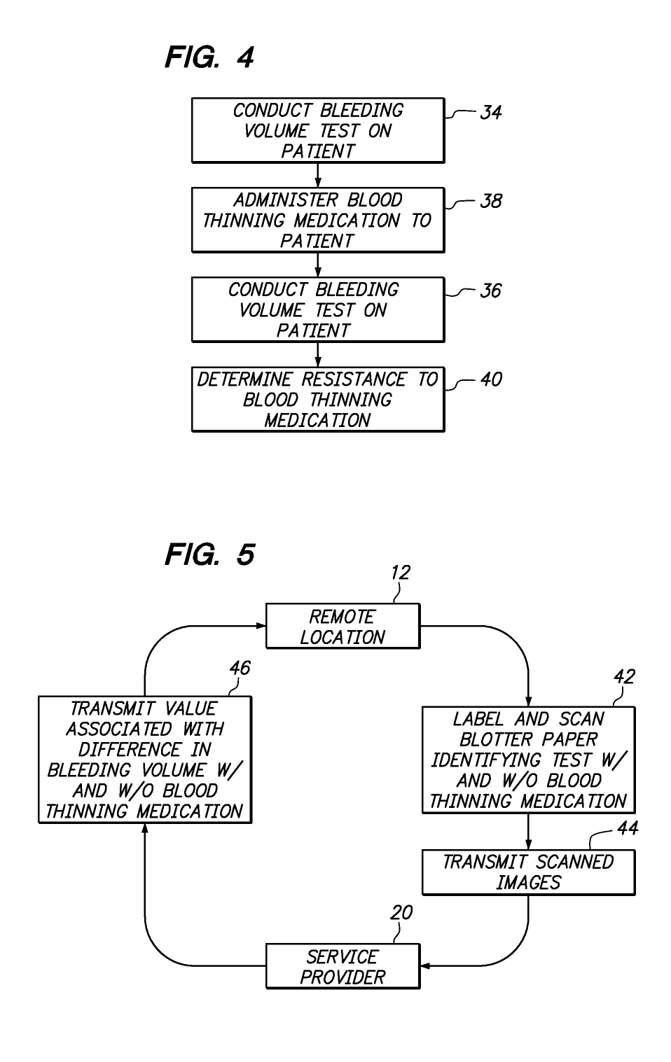

FIGS. 4 and 5 illustrate a method for determining a patient's resistance to a blood thinning medication (e.g., aspirin). By way of example and not limitation, the blood thinning medication may be aspirin, the ADP P2Y₁₂ receptor blocker: clopidogrel, GPIIb-IIIa antagonists: abciximab, tirofiban and eptifibatide, etc. As shown in FIG. 1, an incision 18 is made on a patient 10 prior to any administration of a blood thinning medication to the patient 10. A first pretreatment set of blotter paper 14 is used to absorb the blood 16 oozing out of the incision 18 until the bleeding stops. Blood thinning medication is now administered to the patient 10. After a period of time has elapsed to allow the blood thinning medication to reach its maximum effect on the patient 10, a post-treatment second incision 18 is made on the patient 10. A second clean set of blotter paper 14 absorbs the blood 16 oozing out of the second incision 18 until the patient stops bleeding. One or more images of the first and second sets of blotter papers 14 may be transmitted to a service provider 20. The service provider may calculate or determine a value associated with the difference or ratio in the bleeding volume and bleeding time or some function of the patient 10 before and after administration of the blood thinning medication. The service provider 20 may then transmit such value or values back to the medical professional at a remote location.

The value (e.g., difference or ratio of some function of the bleeding volume and bleeding time before and after administration of the blood thinning medication) may be used to gauge the patient's resistance to the blood thinning medication. The medical professional can now make an educated decision as to whether prescribing the blood thinning medication to the patient 10 would be beneficial in light of the risk of the blood thinning medication. Also, since a patient's resistance to blood thinning medication is multi-factorial with possible explanatory factors including multiple genes, variable degrees of blood thinning medication affinity to platelet surface binding sites, variable degrees of cyclooxygenase binding and interaction with blood thinning medication (e.g., aspirin) and variable degrees of blood thinning medication (e.g., aspirin) by availability and metabolism, the test described herein is an in vivo test so as to take into consideration the multi-factorial factors to blood thinning medication resistance. Additionally, the methods and apparatus described herein allows a medical professional at a remote location to gauge the patient's excessive bleeding tendency and blood thinning medication resistance as long as the medical professional has access to a scanner and a means for transmitting the scanned image to the service provider 20 such as through the internet for the purpose of better treating a particular patient.

The method and system disclosed herein may be less expensive, have fewer moving parts, be more accessible almost anywhere in the world and is an in-vivo rather than in-vitro test. Some of the desirable features include but are not limited to the following. The method and system may be less expensive than current diagnostic clinical laboratory devices which as discussed in the background may cost more than $9,000 plus the disposable supplies. The method and system disclosed herein does not require a large capital expenditure and therefore is more readily available for use by impoverished clinics in underdeveloped countries or isolated communities. The test may also available twenty-four (24) hours a day, seven days a week and three-hundred-sixty-five (365) days a year since an embodiment of the method and system contemplates an internet based or supported test. No perishable reagents, expensive cartridges or other expensive disposable supplies are necessary for implementing the method and system disclosed herein. A blotter paper, scanner and means of communication with the service provider 20 may be all that is necessary for implementing the method and test disclosed herein. The service provider 20 provides the back end support to reduce the front end costs to the medical professional at the remote location. The method and system disclosed herein also does not require puncture of a vein (phlebotomy) for obtaining a blood sample which in some patients may be problematic. Many older patients and obese patients may have inaccessible veins. Since an embodiment of the method and system disclosed herein is an online platform with which medical professionals interact, the same allows an easy creation of a clinical database for collecting and storing large amounts of data as well as proper security and confidentiality. Additionally, the online aspect of the procedure and device or system allows continual updating of clinical databases containing a wide variety and large number of clinical covariates (i.e., associated common medical conditions and rare diseases). This allows every participating clinician or medical professional to compare an individual patient to a highly relevant set of patients with similar co-morbidities. This may significantly increase the test's sensitivity and specificity.

The online bleeding volume test allows for the screening of platelet dysfunction, diagnostic test for Von Willebrand Disease, monitoring anti-platelet therapy with aspirin, clopidogrel, test of compliance, predicting bleeding, detecting platelet hyperfunction and prediction of thrombosis, platelet function testing in transfusion medicine. Additionally, the website supporting the online bleeding volume test may facilitate large prospective studies.

Referring now to FIG. 1, a medical professional conducts the following procedure on the patient 10 being treated. A blood pressure (e.g., sphygmomanometer) cuff 26 may be placed on an upper arm 22 of the patient 10. By way of example and not limitation, the blood pressure cuff 26 may be inflated to about 40 mm Hg to regulate a constant venous blood pressure in the forearm 24 of the patient 10. After the blood pressure of the patient's forearm 24 is stabilized, which typically takes no more than thirty seconds, a standard dermal incision 18 (e.g., 5 mm long and 1 mm deep) is made in the skin of the volar forearm 24 of the patient 10. A round disc blotter paper 14 typically used for determining bleeding time may be placed at the edge of the incision 18. The blood 16 from the wound is wicked onto the edge of the blotter paper 14. At the end of each thirty second interval, the blotter paper 14 may be rotated slightly and a clean unused edge of the blotter paper 14 is gently touched to the edge of the accumulating droplet of blood 16 at the incision site 18 taking care that adjacent blotches of blood do not intersect. One or more blotter papers 14 may be necessary to absorb all of the blood 16 oozing out of the incision 18. When the bleeding stops, the blood pressure cuff 26 is removed from the patient 10 and the incision 18 is cleaned and bandaged as clinically indicated.

As shown in FIG. 2, the blood 16 absorbed into the blotter paper 14 creates blotches 28 of absorbed blood 16. The aggregated surface area of the blotches 28 formed on the one or more blotter papers 14 during the procedure described above correspond to the bleeding volume. Bleeding volume is defined as the volume of blood that oozes out of the incision 18 from the moment an incision 18 is made to the moment the bleeding stops. The number of blotches 28 corresponds to the bleeding time. Bleeding time is defined as the total length of time between the moment the incision 18 is made and the moment the bleeding stops. In particular, two blotches 28 of absorbed blood correspond to one minute. To calculate the total bleeding time, the number of blotches 28 may be counted and divided by two to arrive at the bleeding time to the nearest thirty (30) second interval. Alternatively, the bleeding time may be calculated with a stopwatch.

To determine the bleeding volume based on the blotches 28 formed on the blotter paper 14, the one or more blotter papers 14 may be scanned as a JPEG (joint photographic experts group) file and emailed 32 (see FIG. 3) to a service provider 20. The service provider 20 then takes the scanned image and determines the bleeding volume. In particular, when the medical professional scans the one or more blotter papers 14, a reference area 30 may be formed on (see solid line in FIG. 2) or alongside (see dash line in FIG. 3) the one or more blotter papers 14. The reference area 30 may be for example 1 cm². When the service provider 20 receives the scanned image, the service provider 20 may determine the number of pixels forming the reference area 30 (i.e., pixels to surface area conversion rate). With this information, the service provider 20 may determine the number of pixels per centimeter squared or its inverse. The service provider 20 may also determine the number of pixels forming the blotches 28. The number of pixels forming the blotches 28 on the blotter papers 14 may be divided by the pixels per centimeter squared (pixel to surface area conversion rate) determined above. This results in the total surface area in centimeter squared of the blotches 28.

With the total surface area of the blotches 28, the service provider 20 may now calculate the total blood volume of the blood forming the blotches 28. To this end, the service provider 20 may take a known volume of blood 16 (e.g., 30 micro liters) and drop the known volume (e.g., 30 micro liters) of blood 16 on blotter paper 14a which may be the same or similar type of blotter paper 14 used by the medical professional. This is shown in FIG. 2A. The service provider 20 can calculate the area of the blotch 28a and determine the surface area per volume or volume per surface area of blood 16 (i.e., area/volume conversion rate) because there is a linear correlation between the volume of blood and the surface area of the blotch 28a. The service provider 20 may then divide the total surface area of the blotches 28 shown in FIG. 2 by the area/volume conversion rate determined by the service provider 20. This produces the total volume of blood representing the bleeding volume of the patient 10. The bleeding volume of the patient 10 can now be transmitted back to the medical professional to further aid the medical professional in diagnosing the patient 10.

Instead of the service provider 20 determining the area/volume conversion rate, the medical professional may aid the service provider 20 in doing so. In particular, the medical professional may utilize an additional blotter paper 14 and drop a known quantity of blood 16 of the patient treated on the blotter paper 14 to form blotch 28b, as shown in FIG. 2 in dash lines. The blotter paper 14 along with the blotch 28b may be scanned alongside the one or more blotter papers 14. The blotter paper 14 with blotches 28, the blotter paper 14 with blotch 28b and the reference surface area 30 may be scanned and transmitted to the service provider 20. The service provider 20 calculates the bleeding volume based on (1) the total surface area formed by the blotches 28, (2) the pixels to surface area conversion rate) and (3) the area/volume conversion rate determined by blotch 28b, as discussed above. The service provider 20 may then retransmit 34 the bleeding volume to the medical professional.

Referring now to FIG. 3, a general overview of the process is shown. The medical professional may be at a remote location 12. The remote location 12 may be a location other than the location of the service provider 20. For example, the remote location 12 may be across town, in a different state, in a different country. The medical professional conducts the procedure discussed above with the incision 18 and the blotter paper 14 at the remote location 12. The blotter paper 14 with blotches 28 may be scanned as an image (e.g., JPEG file) and emailed to the service provider 20. Alternatively, the blotter papers 14 may be photocopied and faxed to the service provider 20. The copying and faxing of the blotter paper 14 alters the scaling of the blotches 28 and 28a, b. However, the reference surface area 30 also changes a corresponding amount. As such, the service provider 20 may determine the bleeding volume based on the faxed 32 blotter papers 14. By way of example and not limitation, the service provider 20 may scan in the fax to determine (1) the number of pixels of the aggregate blotches 28, (2) pixels to surface area conversion rate and (3) the area per volume conversion rate to ultimately calculate the bleeding volume. Once the service provider 20 determines the bleeding volume of the patient 10, the bleeding volume is then transmitted 34 back to the medical professional at the remote location 12. With this information, the medical professional may make a better treatment decision for that particular patient.

Generally, the service provider 20 transmits a value associated with the bleeding volume. By way of example and not limitation, such value may be the bleeding volume, bleeding time and/or bleeding rate. The bleeding rate is defined as the bleeding volume divided by the bleeding time. The value may be represented as a continuous variable in the real numbers, or a discrete subset of the integers. It is also contemplated that the value may be represented as a dichotomous score such as yes/no, sick/well, condition present/absent. Also, the value may be a categorical score such as a number from one (1) to ten (10) wherein one (1) through three (3) represents minimal bleeding tendencies, a number between four (4) through seven (7) represents an average bleeding tendency, and a number eight (8) through ten (10) represents excessive bleeding tendencies. The medical professional may use this value associated with bleeding volume to further diagnose and treat the patient 10.

It is also contemplated that the value associated with the bleeding volume may be derived without calculating the total surface area of the blotches 28. In particular, a number of pixels for all of the blotches 28 is determined from the image file (e.g., JPEG file) provided by the medical professional or by scanning in a fax of the blotter paper 14 and calculating the number of pixels forming the blotches 28. The service provider 20 may calculate the number of pixels forming the blotch 28b of a known volume of blood. The service provider 20 divides the total number of pixels of the blotches 28 by the pixels of the blotch 28b the known volume of blood to derive the total bleeding volume. A value associated with the bleeding volume may be transmitted back to the medical professional such that the medical professional may make a better treatment decision for a particular patient.

Referring now to FIG. 4, a method for determining a blood thinning medication resistance score is shown. In particular, the bleeding volume test of the patient 10 is conducted 34, 36 before and after blood thinning medication is administered 38 to the patient 10. A difference in bleeding volume is determined 40 and used to indicate the patient's resistance to the blood thinning medication. If the patient 10 is resistant to the blood thinning medication, then the blood thinning medication would provide marginal or no benefit to the patient 10 in preventing acute myocardial infarction and/or major stroke yet expose the patient 10 to harmful side effects (e.g., gastric irritation or erosion) of the blood thinning medication. Conversely, if the patient 10 is not resistant to the blood thinning medication, then the benefit received by the patient in preventing acute myocardial infarction and major stroke may outweigh the harmful side effects of the blood thinning medication. By way of example and not limitation, aspirin (a blood thinning medication) has been shown to be effective in preventing acute myocardial infarction and major stroke. However, aspirin may produce side effects such as gastric irritation or erosion. If the patient 10 receives marginal or no benefit in preventing acute myocardial infarction and major stroke by taking aspirin, then there would be little incentive to prescribe a regimen of aspirin since the patient 10 would be exposed to side effects of aspirin such as gastric irritation and erosion without significant benefit. On the other hand, if the patient 10 is not resistant to blood thinning medication, then it may be beneficial to the patient 10 to take blood thinning medication in preventing acute myocardial infarction and major stroke even though the patient 10 is exposed to side effects of the blood thinning medication. Generally, the greater the difference in bleeding volume before and after administration of blood thinning medication indicates less resistance to the blood thinning medication. Conversely, the smaller the difference in bleeding volume before and after administration of the blood thinning medication, then the greater the resistance to the blood thinning medication and less benefits are received by the patient 10 in preventing acute myocardial infarction and/or major stroke.

The blood thinning medication resistance score (hereinafter “RS”) may be characterized as a numerical quantification of the relative degree of resistance to blood thinning medication by the patient 10. To derive the numerical quantification, the bleeding volume test is conducted 34 on the patient 10 prior to administration of the blood thinning medication to the patient 10. Moreover, the medical professional may require that the patient 10 cleanse his/her system from prior doses of blood thinning medication (e.g., aspirin). By way of example and not limitation, the medical professional may instruct the patient 10 to not take aspirin or other blood thinning medications for about one to two weeks. When the patient 10 has cleared his/her system from all blood thinning medication, the medical professional conducts 34 the bleeding volume test on the patient 10. In particular, a blood pressure cuff 26 is placed on the upper arm 22 of the patient 10. The pressure may be set to about 40 mm Hg. The blood pressure in the arm of the patient is stabilized and a standard dermal incision 18 may be made on the forearm 24 of the patient 10. Blotter paper 14 is placed adjacent the accumulating droplets of blood 16 forming adjacent the incision 18. The blotter paper 14 absorbs the blood 16 and forms a blotch 28 of blood 16 on the blotter paper 14. Every thirty seconds, the blotter paper 14 may be rotated such that a clean edge of the blotter paper 14 is used to absorb the accumulating droplets of blood 16 adjacent the incision 18. This process is continued until the patient 10 stops bleeding. This set of blotter paper 14 with blotches 28 is properly identified (e.g., PRIOR TO ASPIRIN ADMINISTRATION) and may be scanned as a single image file (e.g., JPEG file).

The blood pressure cuff 26 is removed from the patient 10. The patient 10 is then directed to ingest 38 a quantity of blood thinning medication (e.g., 325 mg of aspirin). The medical professional may wait approximately one hour or some other period of time before conducting 36 the bleeding volume test of the patient for a second time. The one hour time period allows the blood thinning medication to reach its peak effect on the patient. When the bleeding volume test is conducted 36 on the patient for the second time, the difference in bleeding volume prior to and after administration of the blood thinning medication is at its maximum. After the blood thinning medication has reached its maximum effect on the patient 10, the medical professional re-conducts 36 the bleeding volume test on the patient 10 so as to produce a second set of blotter papers 14 with blotches 28 formed by the blood 16 oozing out of the incision 18 of the patient 10. The first and second sets of blotter paper 14 may be properly labeled and scanned 42 along with a reference area 30. Optionally, a blotch 28b of a known volume of blood 16 of the patient 10 may be formed on a separate blotter paper 14 such that the service provider 20 may determine the pixels per volume conversion rate and/or surface area per volume conversion rate. The scanned images are then transmitted 44 to the service provider 20.

The service provider 20 calculates a value associated with a difference in the bleeding volume. In particular, for each of the sets of blotter paper 14, the bleeding volume of the patient 10 may be determined as discussed above. The number of pixels forming the blotches 28 for the first set of blotter paper 14 may be determined. The number of pixels forming the reference surface area 30 may be calculated. The number of pixels forming the blotches 28 for the first set of blotter paper 14 may be divided by the number of pixels forming the reference surface area 30 to arrive at the total surface area of the blotches 28 for the first set of blotter paper 14. The total surface area 30 of the blotches 28 of the first set of blotter paper 14 may be divided by the area per known volume of blood determined by the blotch 28a or 28b (see FIGS. 2 and 2A). This derives the bleeding volume of the patient 10 prior to administration of the blood thinning medication. The same calculation may be conducted for the second set of blotter paper 14 to arrive at the bleeding volume of the patient 10 after administration of the blood thinning medication. The service provider 20 transmits 46 the value back to the medical professional. The value may be the blood thinning medication resistance score or RS. As previously stated, the medical professional may be located at a remote location 12 such as across town, in a different state, in a different country. As long as the medical professional has access to a scanner and a means for transmitting the scanned images (e.g., fax, internet, email, etc.), the medical professional may obtain more information about the patient he/she is treating to make an educated treatment decision for a particular patient.

The RS may be a function of bleeding volume. By way of example and not limitation, the RS may be defined as (BRₐ−BR₀)/BR₀ or (BRₐ−BR₀)/BRₐ where the dermal bleeding rate BR=BV/BT, BV is dermal bleeding volume, BT=bleeding time, BRₐ is bleeding rate with aspirin or blood thinning medication and BR₀ is bleeding rate without aspirin or blood thinning medication. The RS may alternatively be defined as (BVₐ−BV₀)/BV₀ or (BVₐ−BV₀)/BVₐ where BVₐ is bleeding volume with aspirin or blood thinning medication and BV₀ is bleeding volume without aspirin or blood thinning medication. As a further alternative to the resistance score, the same may be expressed as a difference in BVₐ−BV₀ where BVₐ is bleeding volume with aspirin or blood thinning medication and BV₀ is bleeding volume without aspirin or blood thinning medication. As a further alternative to the RS, the same may be expressed as an abstract number or concept. By way of example and not limitation, the RS may be expressed as average blood thinning medication resistance, highly resistant to blood thinning medication or low resistance to blood thinning medication. Also, the RS may be expressed as a number from one (1) to ten (10) wherein one (1) through three (3) represents low resistance to blood thinning medication, a number between four (4) through seven (7) represents an average resistance to blood thinning medication and a number between eight (8) through ten (10) represents highly resistant to blood thinning medication. The medical professional may use this RS value to made an educated treatment decision for a particular patient 10.

As shown above, the RS may be defined as a unitless number such as (BRₐ−BR₀)/BR₀ or (BVₐ−BV₀)/BV₀. These RS are unitless numbers. As such, the bleeding volume before and after administration of the blood thinning medication is not needed to calculate these RS values. By way of example and not limitation, the formula (BVₐ−BV₀)/BV₀ can also be expressed as the number of pixels forming the blotches 28 for the second set of blotter paper 14 minus the number of pixels forming the blotches 28 for the first set of blotter paper 14, the result of which is divided by the number of pixels forming the blotches 28 in the first set of blotter paper 14. As such, in this calculation, the reference surface area 30 and the blotches 28a, b of a known quantity of blood on the blotter paper 14 is unnecessary. The same pixel based calculation can be done for (BRₐ−BR₀)/BR₀ or (BRₐ−BR₀)/BRₐ or (BVₐ−BV₀)/BVₐ.

The above description is given by way of example, and not limitation. Given the above disclosure, one skilled in the art could devise variations that are within the scope and spirit of the invention disclosed herein, including various ways of transmitting the scanned images. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A method for determining bleeding volume of a patient at a remote location, the method comprising the steps of:
breaking a skin of the patient to induce bleeding;
absorbing blood of the patient on a flat absorbent material until the patient stops bleeding;
providing a reference surface area on the flat absorbent material;
scanning an image of the flat absorbent material including blotches of the absorbed blood and the reference surface area;
transmitting the scanned image to a service provider;
receiving a value associated with the bleeding volume of the patient from the service provider.

2. The method of claim 1 wherein the breaking the skin of the patient includes the step of making an incision on the skin of the patient.

3. The method of claim 1 wherein the flat absorbent material is blotter paper.

4. The method of claim 1 wherein the scanning step includes the step of creating a portable document file or picture file of the flat absorbent material.

5. The method of claim 1 wherein the transmitting step includes the step of emailing the scanned image to the service provider.

6. The method of claim 1 wherein the scanning step and the transmitting step includes the step of faxing an image of the flat absorbent material to the service provider.

7. A method for determining bleeding volume of a patient at a remote location, the method comprising the steps of:
receiving a scanned image of a flat absorbent material including absorbed blood of the patient and a reference surface area;
determining the area of absorbed blood;
determining a value associated with the bleeding volume of the patient as a function of the area of absorbed blood and the reference surface area;
transmitting the value to the remote location.

8. The method of claim 7 wherein the determining the area of the absorbed blood step includes the steps of:
determining a number of pixels of the absorbed blood on the flat absorbent material;
determining a number of pixels of the reference surface area;
dividing the number of pixels of the absorbed blood by the number of pixels of the reference surface area.

9. The method of claim 7 wherein the transmitting step includes the step of faxing, emailing or calling.

10. A method of determining resistance of a patient to blood thinning medication, the method comprising the steps of:
prior to administering blood thinning medication, breaking a skin of the patient to induce bleeding;
absorbing the blood of the patient on a first flat absorbent material until the patient stops bleeding;
administering the blood thinning medication to the patient;
after administering the blood thinning medication to the patient for a period of time so that the blood thinning medication achieves maximum effect, breaking the skin of the patient to induce bleeding;
absorbing the blood of the patient on a second flat absorbent material until the patient stops bleeding;
transmitting one or more images of the first and second flat absorbent material to a service provider; and
receiving a value associated with a difference or ratio in bleeding volume prior to and after administration of the blood thinning medication from the service provider based on the one or more images of the first and second flat absorbent material.

11. The method of claim 10 wherein the administering step includes injecting the patient with the blood thinning medication or directing the patient to orally ingest the blood thinning medication.
